(19) United States
(12) Patent Application Publication
(10) Pub. No.: US 2024/0281964 A1
(43) Pub. Date: Aug. 22, 2024

(54) METHOD AND DATA PROCESSING SYSTEM FOR PROVIDING VERTEBRAL DATA

(71) Applicant: Siemens Healthineers AG, Forchheim (DE)

(72) Inventor: Lutz GUENDEL, Erlangen (DE)

(73) Assignee: Siemens Healthineers AG, Forchheim (DE)

(21) Appl. No.: 18/441,359

(22) Filed: Feb. 14, 2024

(30) Foreign Application Priority Data
Feb. 16, 2023 (DE) 1020232013487

(51) Int. Cl. G06T 7/00; G06T 7/10; G06T 7/66; G16H 30/40

(52) U.S. Cl. CPC G06T 7/0012; G06T 7/10; G06T 7/66; G16H 30/40; G06T 2207/30012

(57) ABSTRACT

A computer-implemented method for providing vertebral data, comprises: receiving imaging data of an examination region, wherein the examination region has a set of vertebrae from a vertebral column; calculating a representation of the set of vertebrae based on the imaging data; calculating the vertebral data based on the representation of the set of vertebrae; and providing the vertebral data.

### CROSS-REFERENCE TO RELATED APPLICATION (S)

The present application claims priority under 35 U.S.C. § 119 to German Patent Application No. 10 2023 201 348.7, filed Feb. 16, 2023, the entire contents of which is incorporated herein by reference.

### FIELD

One or more example embodiments of the present invention relate to a method for providing vertebral data. One or more example embodiments of the present invention further relate to a data processing system for providing vertebral data.

### BACKGROUND

A healthy vertebral column typically has a double-s shape when projected into a sagittal plane and ideally a straight shape when projected into a frontal plane. Typical diseases of the vertebral column result in a lateral deviation of the vertebral column, however, often accompanied by twisting. In severe cases, the vertebral bodies can be deformed. The twisting is often reciprocally opposed, since this is necessary for the body to maintain equilibrium.

### SUMMARY

In order to examine the vertebral column, x-ray images can be generated for both the frontal plane and the sagittal plane. Angles and/or lengths can be manually measured on these images, e.g. via corresponding graphical tools. A quantitative detection of scoliosis can be effected on the basis of the Cobb angle, for example, and the inflexion points of the lateral curvature of the vertebral column in the frontal plane can be used to ascertain this. A plurality of methods exist for ascertaining rotation of the vertebral column, e.g. as per Nash and Moe, Perdriolle or Raimondi.

An object of one or more example embodiments of the present invention is to allow an alternative to the conventional provision of vertebral data. At least this object is achieved by the subject matter of an independent claim. Further advantageous aspects of embodiments of the present invention are considered in the dependent claims. Independent of the grammatical term usage, individuals with male, female or other gender identities are included within the term.

An embodiment of the present invention relates to a computer-implemented method for providing vertebral data, which method comprises:
receiving imaging data relating to an examination region, said examination region comprising a set of vertebrae from a vertebral column,
calculating a representation of the set of vertebrae on the basis of the imaging data,
calculating the vertebral data on the basis of the representation of the set of vertebrae,
providing the vertebral data.

The imaging data can be e.g. two-dimensional imaging data and/or three-dimensional imaging data. The imaging data can be e.g. medical imaging data, in particular computed tomography imaging data and/or magnetic resonance imaging data. In particular, it is possible to use imaging data that was not originally recorded for the purpose of examining the vertebral column, but for another purpose, the set of vertebrae from the vertebral column being nonetheless contained in the examination region.

The examination region can comprise the vertebral column or merely a section of the vertebral column. The examination region can be in particular part of a human body and/or comprise e.g. a thoracic cavity, an abdominal cavity and/or a pelvic region. The vertebral column can be in particular a human vertebral column.

In particular, a medical image of the examination region can be calculated on the basis of the imaging data. The medical image can be two-dimensional or three-dimensional, for example. The representation of the set of vertebrae can be visualized in the medical image of the examination region, for example.

The vertebral data can relate to e.g. a distortion of the vertebral column and/or torsion of the vertebral column. The vertebral data can be calculated, in particular automatically, on the basis of the representation of the set of vertebrae. In this way, e.g. clinical features in the set of vertebrae, in particular relating to the arrangement of the vertebrae, can be taken into consideration automatically. The vertebral data can be provided e.g. in a format which is suitable for inclusion in medical findings. In particular, the execution of the method according to an embodiment of the present invention requires neither manual angle measurements nor manual length measurements with subsequent conversion to angles.

An embodiment variant provides for the representation of the set of vertebrae to comprise, for each vertebra in the set of vertebrae, at least two points of a top side of the respective vertebra and/or at least two points of a bottom side of this vertebra. In particular, provision can be made for each vertebra in the set of vertebrae to extend essentially between the top side of the respective vertebra and the bottom side of the respective vertebra, in particular in such a way that the respective vertebra is delimited by the top side of the respective vertebra and the bottom side of the respective vertebra in relation to adjacent vertebrae in the set of vertebrae.

Each vertebra in the set of vertebrae can comprise in particular a vertebral body. In particular, for each vertebra in the set of vertebrae, the vertebral body of the respective vertebra can comprise a top plate and/or a bottom plate. In particular, provision can be made such that, for each vertebra in the set of vertebrae, the vertebral body of the respective vertebra extends essentially between the top plate of the vertebral body of the respective vertebra and the bottom plate of the vertebral body of the respective vertebra, in particular in such a way that the vertebral body of the respective vertebra is delimited by its top plate and its bottom plate in relation to the vertebral bodies of the adjacent vertebrae in the set of vertebrae.

In particular, provision can be made such that, for each vertebra in the set of vertebrae, the at least two points of the top side of the respective vertebra are situated in a region of the top plate of the vertebral body of this vertebra and/or such that, for each vertebra in the set of vertebrae, the at least two points of the bottom side of the respective vertebra are situated in a region of the bottom plate of the vertebral body of this vertebra.

An embodiment variant provides for the representation of the set of vertebrae to be calculated on the basis of the imaging data, specifically by applying a segmentation algorithm to the imaging data. The segmentation algorithm can be based on e.g. a threshold value method, a region growing method and/or on artificial intelligence. The segmentation algorithm can be suitable in particular for vertebral body segmentation and/or vertebra segmentation.

An embodiment variant provides for rotation information to be calculated for each vertebra in the set of vertebrae, said rotation information relating to a rotation of the respective vertebra, on the basis of the representation of the set of vertebrae, the vertebral data for each vertebra in the set of vertebrae comprising the rotation information which relates to the rotation of the respective vertebra.

In particular, provision can be made for calculating a first point of rotation and a first distance point for each vertebra in the set of vertebrae on the basis of the representation of the set of vertebrae, and for calculating the rotation information which relates to the rotation of the respective vertebra on the basis of the first point of rotation and the first distance point. For example, the rotation information can relate to an angle of rotation of the first distance point about the first point of rotation in a first plane of rotation, and in particular comprise a value for the angle of rotation of the first distance point about the first point of rotation in the first plane of rotation.

In particular, provision can be made for calculating a second point of rotation and a second distance point for each vertebra in the set of vertebrae on the basis of the representation of the set of vertebrae, and for calculating the rotation information which relates to the rotation of the respective vertebra on the basis of the second point of rotation and the second distance point. For example, the rotation information can relate to an angle of rotation of the second distance point about the second point of rotation in a second plane of rotation, and in particular comprise a value for the angle of rotation of the second distance point about the second point of rotation in the second plane of rotation.

In particular, provision can be made for the top side of the vertebra to comprise the first point of rotation and the first distance point, and for the bottom side of the vertebra to comprise the second point of rotation and the second distance point. In particular, provision can be made for the first point of rotation and/or the second point of rotation to be situated in a region, in particular a central region, of the vertebral body of the vertebra and/or for the first distance point and/or the second distance point to be situated in a region of a spinous process of the vertebra.

An embodiment variant provides for identification information to be calculated for each vertebra in the set of vertebrae, said identification information relating to an anatomical identification of the respective vertebra, on the basis of the representation of the set of vertebrae, the vertebral data for each vertebra in the set of vertebrae comprising the identification information which relates to the anatomical identification of the respective vertebra.

The identification information which relates to the anatomical identification of a vertebra can be calculated in particular on the basis of the shape and/or the size of the respective vertebra, in particular a vertebral body of this vertebra. For example, algorithms based on artificial intelligence are suitable for the purpose of calculating the identification information and can be trained in both healthy anatomies and pathological changes.

In particular, for each vertebra in the set of vertebrae, the identification information which relates to the anatomical identification of the respective vertebra can comprise a number which is assigned to this vertebra in accordance with an anatomical numbering system. In particular, for each vertebra in the set of vertebrae, the identification information which relates to the anatomical identification of the respective vertebra can specify a section of the vertebral column in which this vertebra is situated, and an index of the vertebra in a sequence of vertebrae from this section of the vertebral column. For example, the number “T10” specifies that this is the tenth vertebra in the thoracic section of the vertebral column.

An embodiment variant provides for a representation of a central line of the set of vertebrae to be calculated on the basis of the representation of the set of vertebrae, the vertebral data being calculated on the basis of the representation of the central line. The central line of the set of vertebrae can be in particular a central line of a section of the vertebral column, said section of the vertebral column comprising the set of vertebrae.

In particular, a support point can be calculated for each vertebra in the set of vertebrae. In particular, the central line can be calculated on the basis of the support points of the vertebrae in the set of vertebrae, e.g. via interpolation between the support points. For example, a spline interpolation is suitable for this purpose. In particular, the curve obtained via interpolation can be smoothed in order to obtain the central line.

The support point for a vertebra can be situated e.g. in a vertebral foramen of the respective vertebra, in particular in the middle of the vertebral foramen of this vertebra. In particular, the central line can essentially run through the vertebral foramina of the vertebrae in the set of vertebrae. Furthermore, for the calculation of the rotation information, provision can be made for the first point of rotation and/or the second point of rotation to be situated on the central line. Furthermore, provision can be made for torsion information relating to a torsion of the vertebral column to be calculated on the basis of the representation of the central line and/or on the basis of the rotation information for the vertebrae in the set of vertebrae, and for the vertebral data to comprise the torsion information.

The representation of the central line can be visualized in the medical image of the examination region, for example. In particular, the representation of the central line can be included in a multiplanar reconstruction of the examination region. The multiplanar reconstruction can be sagittal or coronal, for example.

An embodiment variant provides for distortion information to be calculated for each measurement point in a set of measurement points, said distortion information relating to a deviation of the central line from a reference direction, in particular in the region of the respective measurement point, on the basis of the representation of the central line, the vertebral data for each measurement point in the set of measurement points comprising the distortion information that relates to the deviation of the central line from the reference direction, in particular in the region of the respective measurement point.

The set of measurement points can be calculated on the basis of the representation of the set of vertebrae, for example. The measurement points in the set of measurement points can be arranged along the set of vertebrae and/or along the central line, for example, being distributed in an essentially uniform manner in particular. Each measurement point in the set of measurement points can be arranged between the two vertebral bodies of a corresponding pair of adjacent vertebrae in the set of vertebrae, for example, in particular at a midpoint between the two vertebral bodies of the corresponding pair of adjacent vertebrae in the set of vertebrae.

The distortion information can show in particular a distortion of the vertebral column. The reference direction can be parallel to an anatomical longitudinal axis, for example. The anatomical longitudinal axis can be in particular the anatomical longitudinal axis of the human body which contains the examination region. Furthermore, for the calculation of the rotation information, provision can be made for the first plane of rotation and the second plane of rotation to be perpendicular to the reference direction and/or for the first plane of rotation and the second plane of rotation to be perpendicular to the central line.

An embodiment variant provides for a projection of the central line into a first projection plane to be calculated on the basis of the representation of the central line, the vertebral data being calculated on the basis of the projection of the central line into the first projection plane.

An embodiment variant provides for a projection of a first normal to the curve into the first projection plane to be calculated for each measurement point in the set of measurement points on the basis of the representation of the central line, the first normal to the curve being perpendicular to the central line and parallel to the first projection plane, the distortion information which relates to the deviation of the central line from the reference direction being calculated for each measurement point in the set of measurement points on the basis of the projection of the first normal to the curve into the first projection plane.

An embodiment variant provides for the distortion information which relates to the deviation of the central line from the reference direction to comprise a first item of angle information for each measurement point in the set of measurement points, said first item of angle information relating to an angle between the projection of the first normal to the curve into the first projection plane and an axis which lies in the first projection plane and is perpendicular to the reference direction. In particular, the first item of angle information can comprise a value for the angle between the projection of the first normal to the curve into the first projection plane and the axis which lies in the first projection plane and is perpendicular to the reference direction.

An embodiment variant provides for a projection of the central line into a second projection plane to be calculated on the basis of the representation of the central line, said second projection plane being perpendicular to the first projection plane, the vertebral data being calculated on the basis of the projection of the central line into the second projection plane. In particular, provision can be made for the first projection plane to be parallel to the reference direction and/or for the second projection plane to be parallel to the reference direction. In particular, provision can be made for the first projection plane to be a sagittal plane and/or for the second projection plane to be a frontal plane.

An embodiment variant provides for a projection of a second normal to the curve into the second projection plane to be calculated for each measurement point in the set of measurement points on the basis of the representation of the central line, the second normal to the curve being perpendicular to the central line and parallel to the second projection plane, the distortion information which relates to the deviation of the central line from the reference direction being calculated for each measurement point in the set of measurement points on the basis of the projection of the second normal to the curve into the second projection plane.

In particular, for each measurement point in the set of measurement points, provision can be made for the first normal to the curve and/or the second normal to the curve to be perpendicular to the central line in a region of the respective measurement point. In particular, for each measurement point in the set of measurement points, provision can be made for the first normal to the curve and/or the second normal to the curve to lie in a plane of measurement points which contains the respective measurement point and is perpendicular to the central line.

Furthermore, provision can be made for the distortion information which relates to the deviation of the central line from the reference direction to comprise a second item of angle information for each measurement point in the set of measurement points, said second item of angle information relating to an angle between the projection of the second normal to the curve into the second projection plane and an axis which lies in the second projection plane and is perpendicular to the reference direction. In particular, the second item of angle information can comprise a value for the angle between the projection of the second normal to the curve into the second projection plane and the axis which lies in the second projection plane and is perpendicular to the reference direction.

Embodiments of the present invention further relate to a data processing system for providing vertebral data, having a data interface and a processor, said data processing system being configured to execute one or more embodiments of the inventive method.

Additionally disclosed hereby is a medical imaging system which comprises the inventive data processing system and a medical imaging device for capturing the imaging data of the examination region. The medical imaging device can be a computed tomography device, for example.

The medical imaging device can be selected e.g. from the group of imaging modalities consisting of an x-ray device, a C-arm x-ray device, a computed tomography device (CT device), a molecular imaging device (MI device), a single photon emission computed tomography device (SPECT device), a positron emission tomography device (PET device), a magnetic resonance tomography device (MR device) and combinations thereof, in particular a PET CT device and a PET MR device. The medical imaging device can moreover be a combination of an imaging modality, e.g. selected from the group of imaging modalities, and a radiation modality. In this case, the radiation modality can comprise a radiation unit for the purpose of therapeutic radiation.

Embodiments of the present invention further relate to a non-transitory computer program product comprising instructions which, when the instructions are executed by a computer, cause the computer to execute one or more embodiments of the inventive method.

The computer program product can be a computer

program or comprise at least one further component in addition to the computer program. The at least one further component of the computer program product can take the form of hardware and/or software.

The computer program product can include e.g. a storage medium, on which at least part of the computer program product is stored, and/or a key for authenticating a user of the computer program product, in particular in the form of a dongle. The computer program product and/or the computer program can include e.g. a cloud application program which is designed to distribute the instructions over various processing units, in particular various computers, of a cloud computing system, each of said processing units being designed to execute one or more of the instructions.

Embodiments of the present invention further relate to a non-transitory computer-readable storage medium comprising instructions which, when the instructions are executed by a computer, cause the computer to execute one or more embodiments of the inventive method.

The inventive computer program product can be stored on the computer-readable storage medium, for example. The computer-readable storage medium can be e.g. a memory stick, a fixed disk or other data medium which can be separably connected to a computer or permanently integrated therein. The computer-readable storage medium can take the form of a region within a storage system, for example, the data processing system being connected to the storage system via the data interface.

The data processing system can comprise e.g. one or more components in the form of hardware and/or one or more components in the form of software. The data processing system can at least partially take the form of a cloud computing system. The data processing system can be and/or comprise e.g. a cloud computing system, a computer network, a computer, a tablet computer, a smartphone or similar, or a combination thereof.

The hardware can interact with software and/or be configured by software, for example. The software can be executed by the hardware, for example. The hardware can be e.g. a storage system, an FPGA (field-programmable gate array) system, an ASIC (application-specific integrated circuit) system, a microcontroller system, a processor system and combinations thereof. The processor system can comprise a microprocessor and/or a plurality of interacting microprocessors, for example.

The steps of the method can be executed in the processor of the data processing system, for example, in particular in the form of calculations. A calculation, e.g. the calculation of the representation of the set of vertebrae and/or the calculation of the vertebral data, can be done in particular by applying an algorithm, e.g. a trained machine learning algorithm, to the data on which the calculation is based.

A data transfer between components of the medical imaging system can be effected e.g. via a suitable data transfer interface in each case. The data transfer interface for transferring the data to and/or from a component of the medical imaging system can be realized at least partially in the form of software and/or at least partially in the form of hardware. The data transfer interface can be designed to store data in and/or read data from a region of a storage system, one or more components of the medical imaging system being able to access this region of the storage system.

Data, in particular the imaging data, can be e.g. received by receiving a signal which carries the data and/or by reading in the data, in particular from a computer-readable storage medium. Data, in particular the vertebral data, can be e.g. provided by transmitting a signal which carries the data and/or by writing the data to a computer-readable storage medium and/or by displaying the data on a monitor.

In the context of embodiments of the present invention, features which are described with reference to different embodiment variants of the present invention and/or different statutory classes of claim (method, use, device, system, arrangement, etc.) can be combined to form further embodiment variants of the present invention. For example, a claim relating to a device can also be developed with features which are described or claimed in connection with a method, and vice versa. Functional features of a method can be executed by correspondingly designed material components. The use of the indefinite article “a” or “an” does not preclude multiple instances of the feature concerned. In the context of the present application, the expression “on the basis of” can be understood in particular in the sense of the expression “making use of”. In particular, wording according to which a first feature is calculated (or determined, generated, etc.) on the basis of a second feature, does not preclude the first feature possibly also being calculated (or determined, generated, etc.) on the basis of a third feature.

### BRIEF DESCRIPTION OF THE DRAWINGS

The present invention is explained in the following with reference to exemplary embodiments and to the appended figures. The representation in the figures is schematic, greatly simplified and not necessarily in proportion.

FIG. 1 shows a representation of a set of vertebrae according to a first example.

FIG. 2 shows two projections of a central line of the set of vertebrae according to the first example.

FIG. 3 shows the two projections of the central line of the set of vertebrae in conjunction with angles of rotation of the vertebrae in the set of vertebrae according to the first example.

FIG. 4 shows a representation of a set of vertebrae according to a second example.

FIG. 5 shows two projections of a central line of the set of vertebrae according to the second example.

FIG. 6 shows the two projections of the central line of the set of vertebrae in conjunction with angles of rotation of the vertebrae in the set of vertebrae according to the second example.

FIG. 7 shows a data flow diagram with vertebral data.

FIG. 8 shows a sequence diagram of a method for providing vertebral data.

FIG. 9 shows a medical imaging system.

### DETAILED DESCRIPTION

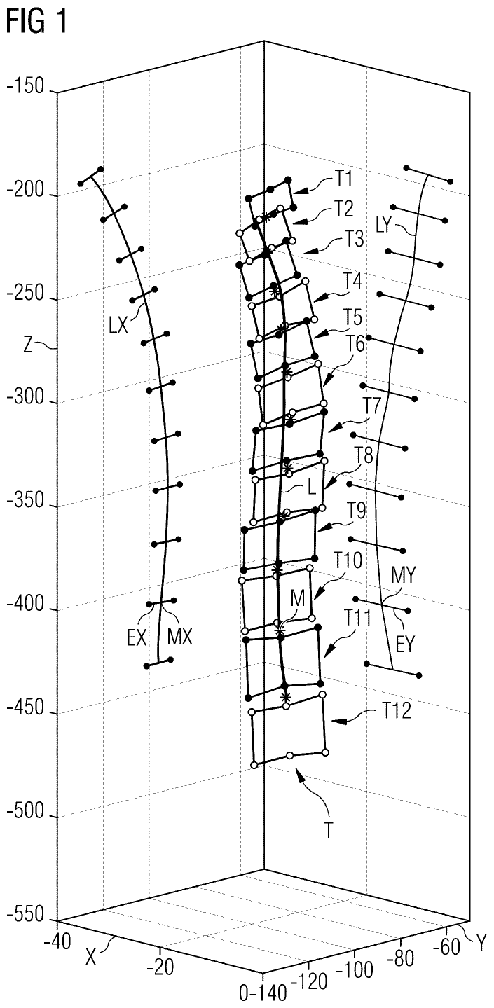

FIG. 1 shows the representation of the set of vertebrae T according to a first example. For each vertebra in the set of vertebrae T according to the first example, identification information relating to an anatomical identification of the respective vertebra is calculated on the basis of the representation of the set of vertebrae T, the vertebral data for each vertebra in the set of vertebrae T comprising the identification information which relates to the anatomical identification of the respective vertebra. The set of vertebrae T according to the first example is part of a thoracic section of a vertebral column of a first patient and comprises the vertebrae T1 to T12. A number is shown for each vertebra in the set of vertebrae T, said number being assigned to the respective vertebra in accordance with an anatomical numbering system. For example, the number “T10” indicates that this is the tenth vertebra of the thoracic section of the vertebral column.

The representation of the set of vertebrae T according to the first example comprises, for each vertebra in the set of vertebrae T, at least two points of a top side of the respective vertebra and at least two points of a bottom side of this vertebra. The representation of the set of vertebrae T according to the first example comprises, for each vertebra in the set of vertebrae T, three points of the top side of the respective vertebra, said points being situated in a region of the top plate of the vertebral body of this vertebra, and three points of the bottom side of the respective vertebra, said points being situated in a region of the bottom plate of the vertebral body of this vertebra. For the purpose of illustration, these six points are connected via a polygonal progression. The two outer points of the top plate of the vertebral body and the midway point of the top plate of the vertebral body do not generally lie on a straight line, because the vertebral body is somewhat smaller in the center than at the edges. The two outer points of the bottom plate of the vertebral body and the midway point of the bottom plate of the vertebral body likewise do not generally lie on a straight line, because the vertebral body is somewhat smaller in the center than at the edges.

A representation of a central line L of the set of vertebrae T is calculated on the basis of the representation of the set of vertebrae T, the vertebral data being calculated on the basis of the representation of the central line L. For each measurement point M in a set of measurement points, distortion information relating to a deviation of the central line L from a reference direction in the region of this measurement point is calculated on the basis of the representation of the central line L, the vertebral data for each measurement point M in the set of measurement points comprising the distortion information that relates to the deviation of the central line L from the reference direction. The reference direction is e.g. parallel to the axis Z.

Each measurement point M in the set of measurement points is e.g. a midpoint between the two vertebral bodies of the corresponding pair of adjacent vertebrae in the set of vertebrae, and is represented as a star. The central line L in the set of vertebrae T is e.g. the interpolated connection between the middles of the vertebral foramina. The central line L does not necessarily lie on the middles of the vertebral bodies or voids.

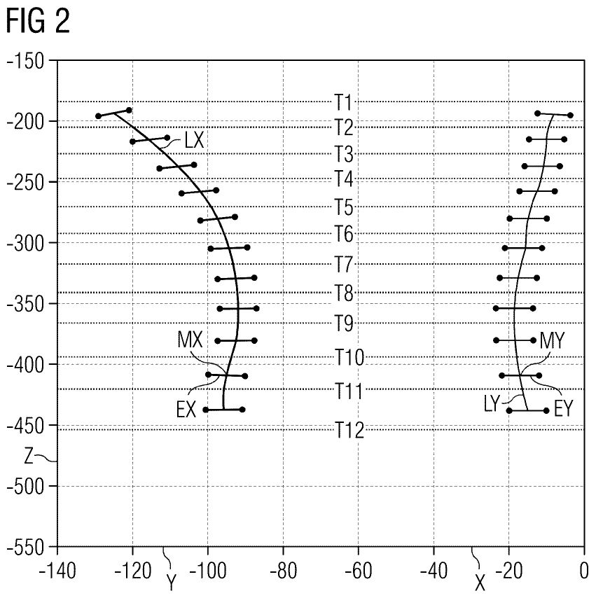

FIG. 2 shows two projections of the central line L of the set of vertebrae T according to the first example. A projection LX of the central line L into a first projection plane is calculated on the basis of the representation of the central line L, the vertebral data being calculated on the basis of the projection LX of the central line L into the first projection plane. A projection LY of the central line L into a second projection plane is calculated on the basis of the representation of the central line L, the second projection plane being perpendicular to the first projection plane, the vertebral data being calculated on the basis of the projection LY of the central line L into the second projection plane. The first projection plane is the sagittal plane, for example. The second projection plane the frontal plane, for example. On the basis of the projection of the central line into the frontal plane, it is possible to ascertain the Cobb angle, for example.

For each measurement point M in the set of measurement points, a projection EX of a first normal to the curve into the first projection plane is calculated on the basis of the representation of the central line L, the first normal to the curve being perpendicular to the central line L and parallel to the first projection plane, the distortion information which relates to the deviation of the central line L from the reference direction being calculated for each measurement point M in the set of measurement points on the basis of the projection EX of the first normal to the curve into the first projection plane.

For each measurement point M in the set of measurement points, a projection EY of a second normal to the curve into the second projection plane is calculated on the basis of the representation of the central line L, the second normal to the curve being perpendicular to the central line L and parallel to the second projection plane, the distortion information which relates to the deviation of the central line L from the reference direction being calculated for each measurement point M in the set of measurement points on the basis of the projection EY of the second normal to the curve into the second projection plane.

For each measurement point M in the set of measurement points, the first normal to the curve and the second normal to the curve lie in a plane of measurement points which contains the respective measurement point and is perpendicular to the central line L. The point MX is the projection of the base point at which the central line L goes through the plane of measurement points that contains the measurement point M, into the first projection plane. The point MY is the projection of the base point at which the central line L goes through the plane of measurement points that contains the measurement point M, into the second projection plane.

For each measurement point M in the set of measurement points, the distortion information which relates to the deviation of the central line L from the reference direction comprises a first item of angle information, said first item of angle information relating to an angle between the projection EX of the first normal to the curve into the first projection plane and an axis Y which lies in the first projection plane and is perpendicular to the reference direction. For each measurement point M in the set of measurement points, the distortion information which relates to the deviation of the central line L from the reference direction comprises a second item of angle information, said second item of angle information relating to an angle between the projection EY of the second normal to the curve into the second projection plane and an axis X which lies in the second projection plane and is perpendicular to the reference direction. As a result of the axial scaling, the angle between the projection EX of the first normal to the curve into the first projection plane and the axis Y which lies in the first projection plane cannot be read easily.

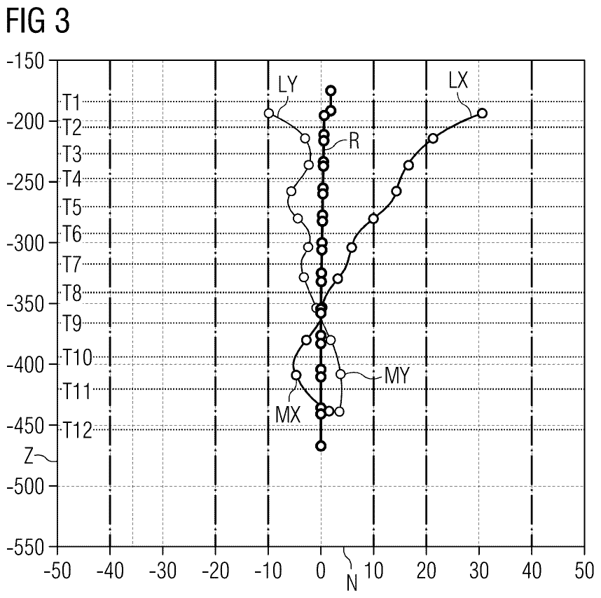

FIG. 3 shows the two projections of the central line L of the set of vertebrae T in conjunction with the angles of rotation R of the vertebrae in the set of vertebrae T according to the first example. Values for the angles of rotation R as well as values for the angles from the first item of angle information (in the sagittal plane) and the second item of angle information (in the frontal plane) can be read on the axis N. For each vertebra in the set of vertebrae T, rotation information which relates to a rotation of the respective vertebra is calculated on the basis of the representation of the set of vertebrae T, the vertebral data for each vertebra in the set of vertebrae T comprising the rotation information which relates to the rotation of the vertebra concerned.

For each measurement point M in the set of measurement points, the corresponding first item of angle information comprises a value for the angle between the projection EX of the first normal to the curve into the first projection plane and the axis Y which lies in the first projection plane and is perpendicular to the reference direction. For each measurement point M in the set of measurement points, the corresponding second item of angle information comprises a value for the angle between the projection EY of the second normal to the curve into the second projection plane and the axis X which lies in the second projection plane and is perpendicular to the reference direction.

It is noticeable here that the deviation of the central line from the reference direction varies in the range from −10° to +4° in the frontal plane. A classification of a mild scoliosis is established in the vertebra T1 accordingly. In the sagittal plane, however, there is a deviation of more than 30° in the vertebra T1. This is significantly more pronounced than in the frontal plane. The angles of rotation R are very small, having a maximum of 1.8° in the vertebra T1.

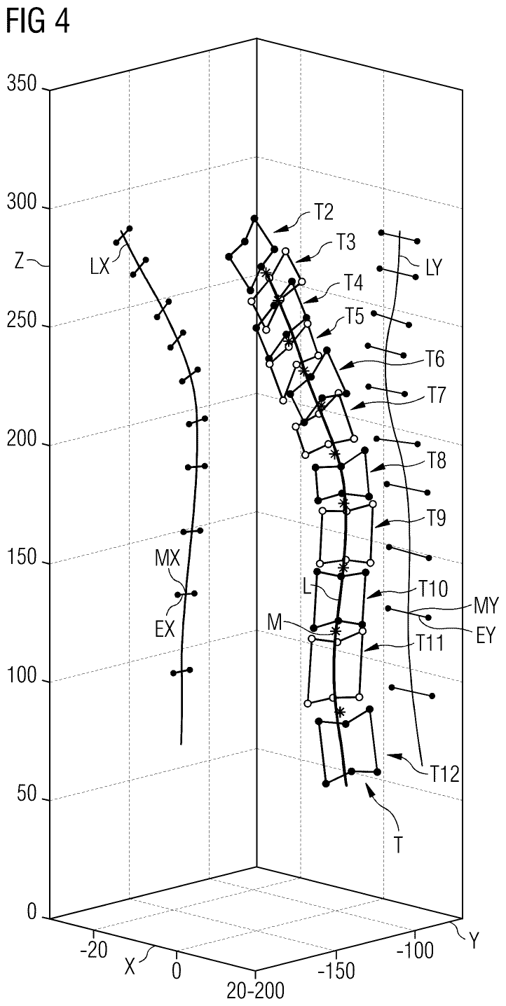

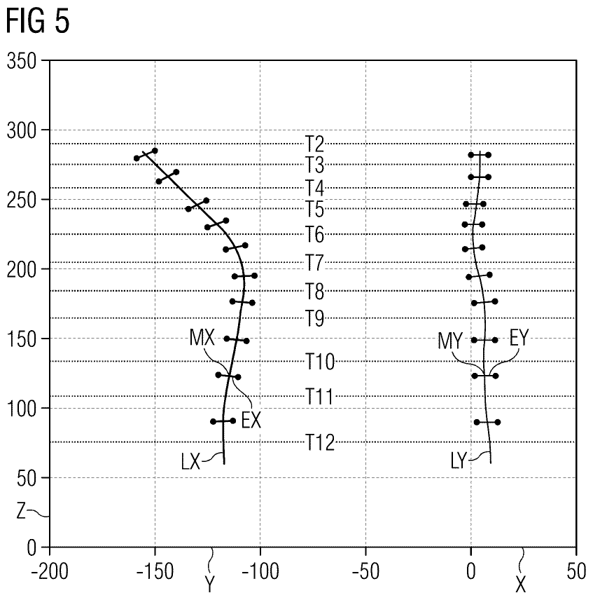

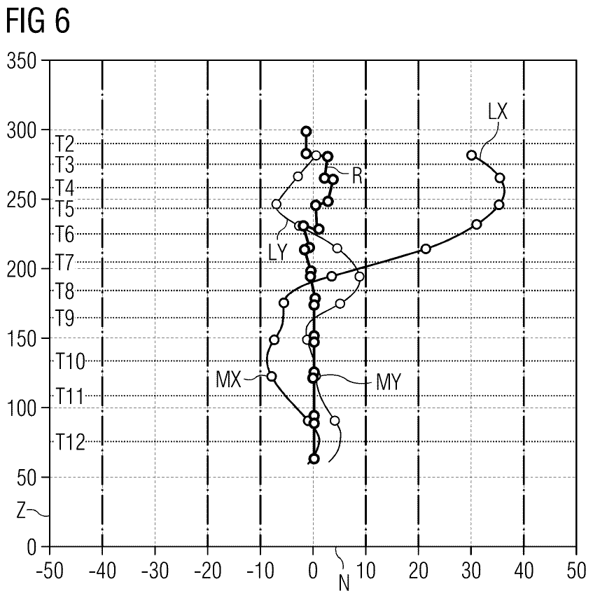

FIG. 4 shows the representation of the set of vertebrae T according to a second example. In this case, the vertebra T1 is not included in the region of measurement. The set of vertebrae T according to the second example is part of a thoracic section of a vertebral column of a second patient. FIG. 5 shows two projections of the central line L of the set of vertebrae T according to the second example. FIG. 6 shows the two projections of the central line L of the set of vertebrae T in conjunction with the angles of rotation R of the vertebrae in the set of vertebrae T according to the second example.

Particularly marked deviations of the central line from the reference direction are evident in the region of the vertebrae T2 to T5. The angular difference between the vertebra T4 and the vertebra T7 in the frontal plane corresponds to the Cobb angle, being 15.6° here. This means that a mild form of scoliosis is present here, but extends over a larger region of the thoracic vertebral column. The angles in the sagittal plane cover an angular range from −8° to +35° with a difference of 43.4º between the vertebra T3 and the vertebra T10. The angles of rotation R are greatest at the vertebral body T4, specifically 3.6°.

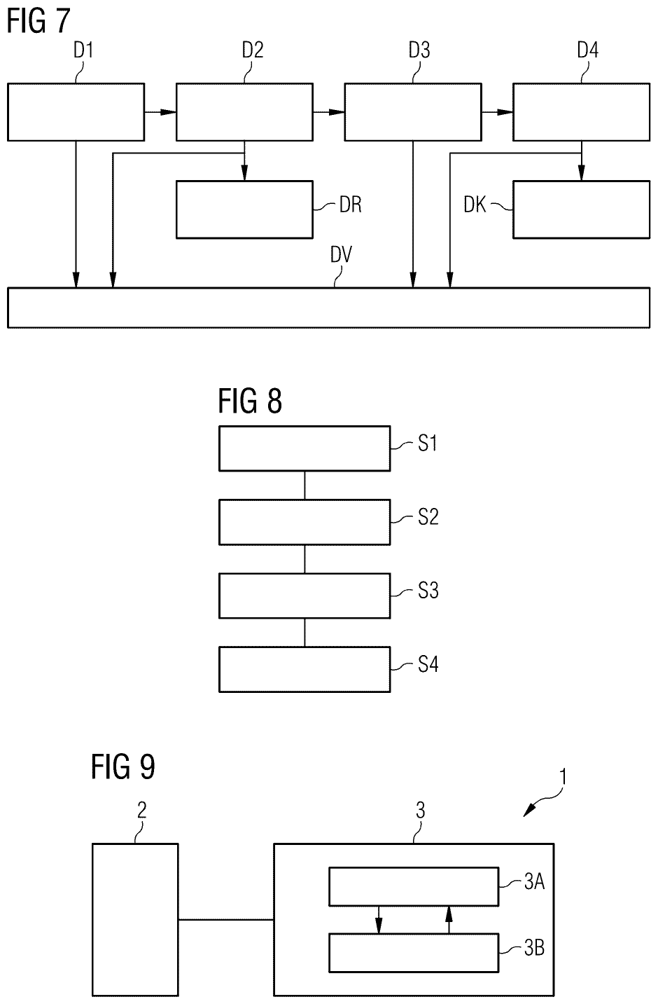

FIG. 7 shows a data flow diagram with vertebral data. The data record DI comprises the imaging data of the examination region, said examination region including the set of vertebrae T in the vertebral column. The data record D2 comprises the representation of the set of vertebrae T, said representation being calculated on the basis of the imaging data. The data record D3 comprises the identification information for each vertebra in the set of vertebrae T, said identification information relating to an anatomical identification of the respective vertebra and being calculated on the basis of the representation of the set of vertebrae T. The data record D4 comprises the representation of the central line L of the set of vertebrae T, said central line L being calculated on the basis of the representation of the set of vertebrae T.

The data record DR comprises the rotation information for each vertebra in the set of vertebrae T, said rotation information relating to a rotation of the respective vertebra and being calculated on the basis of the representation of the set of vertebrae T. The data record DK comprises distortion information for each measurement point M in the set of measurement points, said distortion information relating to a deviation of the central line L from a reference direction and being calculated on the basis of the representation of the central line L. The data record DV comprises an image data record, e.g. a three-dimensional image data record, in which the set of vertebrae T is visualized in conjunction with the identification information and the central line L. In this example, the vertebral data comprises the data records D3, D4, DR, DK and DV.

FIG. 8 shows a sequence diagram of the method for providing vertebral data, which method comprises:
receiving S1 imaging data of an examination region, said examination region comprising a set of vertebrae T from a vertebral column,
calculating S2 a representation of the set of vertebrae T on the basis of the imaging data,
calculating S3 the vertebral data on the basis of the representation of the set of vertebrae T,
providing S4 the vertebral data.

FIG. 9 shows the medical imaging system 1, comprising the data processing system 3 for providing vertebral data and the medical imaging device 2 for capturing the imaging data of the examination region. The data processing system 3 comprises the data interface 3A and the processor 3B and is configured to execute the method according to FIG. 8.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or, ” includes any and all combinations of one or more of the associated listed items. The phrase “at least one of” has the same meaning as “and/or”.

Spatially relative terms, such as “beneath, ” “below, ” “lower, ” “under, ” “above, ” “upper,” and the like, may be used herein for ease of description to describe one element or feature's relationship to another element (s) or feature (s) as illustrated in the figures. It will be understood that the spatially relative terms are intended to encompass different orientations of the device in use or operation in addition to the orientation depicted in the figures. For example, if the device in the figures is turned over, elements described as “below, ” “beneath, ” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on, ” “connected, ” “engaged, ” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A computer-implemented method for providing vertebral data, the computer-implemented method comprising:
receiving imaging data of an examination region, wherein the examination region includes a set of vertebrae from a vertebral column;
calculating a representation of the set of vertebrae based on the imaging data;
calculating the vertebral data based on the representation of the set of vertebrae; and
providing the vertebral data.

2. The method as claimed in claim 1,
wherein the representation of the set of vertebrae includes, for each respective vertebra in the set of vertebrae, at least one of (i) at least two points of a top side of the respective vertebra or (ii) at least two points of a bottom side of the respective vertebra.

3. The method as claimed in claim 1,
wherein the representation of the set of vertebrae is calculated based on the imaging data by applying a segmentation algorithm to the imaging data.

4. The method as claimed in claim 1,
wherein, for each respective vertebra in the set of vertebrae, rotation information relating to a rotation of the respective vertebra is calculated based on the representation of the set of vertebrae, and
wherein the vertebral data for each respective vertebra in the set of vertebrae includes the rotation information relating to the rotation of the respective vertebra.

5. The method as claimed in claim 1,
wherein, for each respective vertebra in the set of vertebrae, identification information relating to an anatomical identification of the respective vertebra is calculated based on the representation of the set of vertebrae, and
wherein the vertebral data for each respective vertebra in the set of vertebrae includes the identification information relating to the anatomical identification of the respective vertebra.

6. The method as claimed in claim 1,
wherein a representation of a central line of the set of vertebrae is calculated based on the representation of the set of vertebrae, and
wherein the vertebral data is calculated based on the representation of the central line.

7. The method as claimed in claim 6,
wherein, for each measurement point in a set of measurement points, distortion information relating to a deviation of the central line from a reference direction is calculated based on the representation of the central line, and
wherein the vertebral data for each measurement point in the set of measurement points includes the distortion information relating to the deviation of the central line from the reference direction.

8. The method as claimed in claim 6,
wherein a projection of the central line into first projection plane is calculated based on the representation of the central line, and
wherein the vertebral data is calculated based on the projection of the central line into the first projection plane.

9. The method as claimed in claim 8,
wherein, for each measurement point in a set of measurement points, a projection of a first normal to the curve into the first projection plane is calculated based on the representation of the central line,
wherein the first normal to the curve is perpendicular to the central line and parallel to the first projection plane, and
wherein, for each measurement point in the set of measurement points, distortion information relating to a deviation of the central line from a reference direction is calculated based on the projection of the first normal to the curve into the first projection plane.

10. The method as claimed in claim 9,
wherein, for each measurement point in the set of measurement points, the distortion information relating to the deviation of the central line from the reference direction includes a first item of angle information, and
wherein the first item of angle information relates to an angle between the projection of the first normal to the curve into the first projection plane and an axis which lies in the first projection plane and is perpendicular to the reference direction.

11. The method as claimed in claim 8,
wherein a projection of the central line into a second projection plane is calculated based on the representation of the central line,
wherein the second projection plane is perpendicular to the first projection plane, and
wherein the vertebral data is calculated based on the projection of the central line into the second projection plane.

12. The method as claimed in claim 11,
wherein, for each measurement point in a set of measurement points, a projection of a second normal to the curve into the second projection plane is calculated based on the representation of the central line,
wherein the second normal to the curve is perpendicular to the central line and parallel to the second projection plane, and
wherein, for each measurement point in the set of measurement points, distortion information relating to a deviation of the central line from a reference direction is calculated based on the projection of the second normal to the curve into the second projection plane.

13. A data processing system for providing vertebral data, the data processing system having a data interface and a processor, wherein the data processing system is configured to execute a method as claimed in claim 1.

14. A non-transitory computer program product comprising instructions which, when executed by a computer, cause the computer to execute a method as claimed in claim 1.

15. A non-transitory computer-readable storage medium comprising instructions which, when executed by a computer, cause the computer to execute a method as claimed in claim 1.

16. The method as claimed in claim 2,
wherein, for each respective vertebra in the set of vertebrae, rotation information relating to a rotation of the respective vertebra is calculated based on the representation of the set of vertebrae, and
wherein the vertebral data for each respective vertebra in the set of vertebrae includes the rotation information relating to the rotation of the respective vertebra.

17. The method as claimed in claim 3,
wherein, for each respective vertebra in the set of vertebrae, rotation information relating to a rotation of the respective vertebra is calculated based on the representation of the set of vertebrae, and
wherein the vertebral data for each respective vertebra in the set of vertebrae includes the rotation information relating to the rotation of the respective vertebra.

18. The method as claimed in claim 4,
wherein, for each respective vertebra in the set of vertebrae, identification information relating to an anatomical identification of the respective vertebra is calculated based on the representation of the set of vertebrae, and
wherein the vertebral data for each respective vertebra in the set of vertebrae includes the identification information relating to the anatomical identification of the respective vertebra.

19. The method as claimed in claim 4,
wherein a representation of a central line of the set of vertebrae is calculated based on the representation of the set of vertebrae, and
wherein the vertebral data is calculated based on the representation of the central line.

20. The method as claimed in claim 19,
wherein, for each measurement point in a set of measurement points, distortion information relating to a deviation of the central line from a reference direction is calculated based on the representation of the central line, and
wherein the vertebral data for each measurement point in the set of measurement points includes the distortion information relating to the deviation of the central line from the reference direction.
